(19) United States
(12) Patent Application Publication
(10) Pub. No.: US 2010/0183718 A1
(43) Pub. Date: Jul. 22, 2010

(54) COMPOSITIONS AND TREATMENT OF HEART DEFICIENCY IN NON-HUMAN ANIMALS

(75) Inventors: Patricia Ovaert, Libourne (FR); Florence Bernay, Leawood, KS (US); Jerome Guyonnet, Ambares (FR)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(73) Assignee: CEVA SANTE ANIMALE SA, LIBOURNE CEDEX (FR)

(21) Appl. No.: 12/666,026

(22) PCT Filed: Jun. 25, 2008
(86) PCT No.: PCT/EP08/58054

§ 371 Date: Dec. 22, 2009

Related U.S. Application Data
(60) Provisional application No. 61/048,419, filed on Apr. 28, 2008.

(30) Foreign Application Priority Data
Jun. 26, 2007 (FR) 0704584

(51) Int. Cl. A61K 9/36; A61K 31/585; A61K 38/05; A61K 31/675; A61K 38/08; A61P 9/04

(52) U.S. Cl. 424/479; 514/173; 514/171; 514/19; 514/91; 514/16

(57) ABSTRACT

The invention relates to novel compositions including an aldosterone antagonist and administered according to a predetermined dosage for treating heart deficiency in non-human mammals.

The invention relates to new compositions comprising an aldosterone antagonist according to a particular posology for the treatment of heart failure in non-human mammal animals.

Heart diseases are frequent in non-human mammals, such as dogs and cats. These may generate heart failure. Heart failure corresponds to a syndrome wherein an anomaly of the heart function causes in the short-term incapacity of the heart to ensure sufficient blood flow rate for covering the energy requirements of the system. This failure may reflect a contraction anomaly of the ventricular cardiac muscle (systolic dysfunction) or a heart filling anomaly (diastolic dysfunction), possibly both these mechanisms.

In veterinary medicine, the severity of heart failure is assessed on the functional aspect according to, among other things, the ISACHC classification (International Small Animal Cardiac Health Council) into three classes. Class I, so-called asymptomatic, is only detectable due to the presence of signs cardiopathy observation during examination, such as for instance cardiac murmur or cardiomegaly. Class II corresponds to mild or moderate heart failure; It is detected by the occurrence of congestive symptoms after effort. Class III corresponds to an advanced or severe heart failure and is reflected by the occurrence of clinical symptoms even at rest, with the presence of ascite and pulmonary oedema.

As the disease starts to develop, the cardiac function is maintained by compensatory mechanisms, whereof the renin-angiotensin-aldosterone system (RAAS) is one of the most important, thanks to its primordial role in volemy maintenance. RAAS is in fact a cardiorenal endocrinic regulation for maintaining salt and water homeostasy of the system, i.e. the balance between electrolytes (sodium ion (Na+), potassium ion (K+), magnesium ion (Mg2+)) and water. It operates by a cascade of endocrinic and enzymatic regulations. The activation of the RAAS starts with the secretion of an enzyme in the kidney, renin, when the pressure drops in the renal artery. However, there are other stimuli, such as the drop in natremia near the bent distal tube or the stimulation of the juxtaglomerular cells by the beta-adrenergic system. Renin cleaves the angiotensinogen which is secreted by the liver, to provide an inactive decapeptide called angiotensin I. angiotensin I is then transformed into angiotensin II mainly near the lung by the angiotensin converting enzyme (ACE). angiotensin II will bond to its transmembranar receptors and favour the rise in arterial pressure through different mechanisms. Angiotensin II has in particular a powerful vasoconstrictor effect on arterioles, it stimulates the secretion of aldosterone, a hormone secreted by surrenal glands which causes an increase in volemy by re-absorption of sodium and water near the kidneys, in the bent distal tube and the collector tube, it also stimulates the secretion of vasopressin, an antidiuretic hormone, which limits the loss of water in urines, and finally inhibits in return the secretion of renin.

During heart failure, the synthesis of aldosterone is increased further to the activation of the renin-angiotensin system. Aldosterone, a mineralocorticoid hormone synthetised by the surrenal glands, the heart, the blood vessels and the brain, operates while bonding to mineralcorticoid receptors. The main biological effects of aldosterone are:
on the kidney, stimulated re-absorption of sodium and water and excretion of potassium and magnesium. The consequence is an increase in volemy.
on the heart and on the vessels, a direct action leading to the tissular remodelling of the myocard and the vascular endothelium as well as the development of fibrosis in the myocard. These effects depend on the bonding of aldosterone to the mineralcorticoid receptors.

In human medicine, the standard therapies of heart failure used are among others the angiotensin converting enzyme inhibitors (ACEI), beta blockers, diuretics, vasodilators, inotropes, digitalic drugs, and hypertensors. The Angiotensin Converting Enzyme inhibitors (ACEI) such as captopril, enalapril, benazepril, lisinopril, or ramipril, enable to regulate the cascade of the RAAS and thus the arterial pressure. Numerous large-scale clinical tests have enabled to demonstrate the efficiency of CEIs: they enable to increase the survival rate significantly in case of heart failure. Conversely, they present counter-indications such as in particular hyperkalemia.

The standard treatment of human heart failure consists of the combination of a CEI and of a diuretic. Numerous diuretics are currently available: ansa diuretics such as furosemide, torsemide, bumetanide, thiazidic diuretics, such as hydrochlorothiazide or chlortalidone, or still potassium savers, such as triamterene, amiloride, eplerenone, and spironolactone.

The effects of an aldosterone antagonist, spironolactone, have also been assessed during heart failure. Experimental studies have demonstrated the deleterious effects of aldosterone on the kidney and the cardiovascular apparatus previously. Tests have hence been realised while relying on the hypothesis that blocking the effects of aldosterone might have beneficial effects during heart failure, and in particular enable to improve the cardiac function and to reduce the incidence of the rhythm disorders.

It has been shown in human, that when spironolactone is administered at doses with diuretic effect (≧50 mg), solely or in combination with a CEI, it exhibits hyperkalemia side effects incompatible with the treatment of heart failure and in particular CEIs. The multicentre clinical study RALES (Randomized Aldactone Evaluation Study) has demonstrated the clinical benefit of spironolactone used at low, so-called sub-diuretic, doses in human patients affected by heart failure. More accurately, the RALES study describes the use of spironolactone at sub-diuretic doses of 1 to 25 mg/day and of a CEI for the treatment of human heart failure. Such combinations are also described in the international application WO 96/24373 and the patent EP 808172B1. Within the framework of the RALES study, 1663 patients affected by severe heart failure, having a left ventricular ejection fraction smaller than 35%, treated with a CEI, ansa diuretics and sometimes with digoxin, have been included in a double-blind, placebo-controlled study. 822 have received 25 mg spironolactone and 841 received a placebo. The study reports 386 dead in the placebo group (46%), against 284 in the spironolactone group (35%). The survival analysis shows that the risk of mortality is reduced by 30% in patients receiving spironolactone relative to a placebo group.

If the administration of spironolactone was known to increase the risk of hyperkaliemia in human subjects affected by heart failure, it is now established that only small daily doses of spironolactone may be used for these treatments. They provide somehow a compromise for reducing the effect of aldosterone on the physiopathology of heart failure while avoiding side effects, in particular hyperkaliemia, due to high dosages of the aldosterone antagonist. It has hence been established that, in human patients, the efficient therapeutic doses of spironolactone sufficient for observing a protective effect, but rather low in view of diuretic doses, must be generally 12.5 to 50 mg per day and per human patient. However and contrary to what had been established previously in terms of dosage of aldosterone antagonists for the treatment of human heart failure, it has been discovered that the administration of doses of aldosterone antagonists greater than the doses used previously a sub-group of patients or particular subjects, constituted of non-human mammal animals, reduced quite significantly the risks of mortality and/or of morbidity, and this without inducing any significant variation in kaliemia or with small variations in kaliemia in these subjects. Indeed, surprisingly, no hyperkaliemia side effect has been observed in non-human mammal animals having received large doses of aldosterone antagonists. Conversely, a greater efficiency unexpected in terms of survival has been demonstrated with a significant reduction in the risks of mortality and morbidity.

### SUMMARY OF THE INVENTION

The present invention hence relates to a new veterinary composition comprising an aldosterone antagonist administered according to a prescribed posology, and a pharmaceutically acceptable vehicle, intended for the treatment of non-human mammal subjects affected by heart failure.

More accurately, the posology or efficient therapeutic dose of aldosterone antagonist is greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take. This therapeutic dose may be associated with an efficient therapeutic dose of a standard therapy for heart failure, such as CEIs, angiotensin II AT-1-receptor antagonists (ARA-II or sartans), digitalic drugs, inotropes, inodilators, diuretics, vasodilators, beta blockers and/or calcic antagonists.

The compositions according to the present invention are particularly useful for the treatment and/or the prevention of heart failure in non-human mammal animals, such as dogs, cats and horses, and generally all pets. They enable in particular to reduce the risk of mortality and/or of morbidity, without causing hyperkaliemia side effects. It has been deducted that the risk of mortality is reduced by 50 to 80%, by 55 to 80%, by 60 to 80%, by 65 to 80%, by 70 to 80%, or by 75 to 80% in dogs receiving spironolactone, its derivatives, or its metabolites, at the posology prescribed according to the invention relative to a placebo group.

The present invention also relates to kits for veterinary usage intended for the treatment of non-human mammal subjects affected by heart failure, having at least one compartment for a separated packaging or not, of daily doses of aldosterone antagonist only or in association with a standard heart failure therapy, such as CEIs, angiotensin II AT-1-receptor antagonists, digitalic drugs, inotropes, inodilators, diuretics, vasodilators, beta blockers and/or calcic antagonists. The compartment may thus contain a daily dose of aldosterone antagonist greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

Moreover, the present invention relates to the use of efficient therapeutic quantities of an aldosterone antagonist solely or in combination with CEIs, angiotensin II AT-1-receptor antagonists, digitalic drugs, inotropes, inodilators, diuretics, vasodilators, beta blockers and/or calcic antagonists, in view of preparing a veterinary medication intended for treating and/or preventing heart failure and/or reducing the rates of mortality and/or of morbidity of non-human mammal animals affected by heart failure, without causing hyperkaliemia side effects. The efficient therapeutic dose of aldosterone antagonist is greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

Another object of the present invention consists of a method for treating early stages of heart failure in non-human mammals comprising the administration of efficient therapeutic doses of an aldosterone antagonist greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

Finally, a last object of the present invention consists of a method for reducing the rates of mortality and/or of morbidity of the non-human mammal animal subjects affected by heart failure comprising the administration of efficient therapeutic doses of an aldosterone antagonist solely or in combination with CEIs, angiotensin II AT-1-receptor antagonists, digitalic drugs, inotropes, inodilators, diuretics, vasodilators, beta blockers and/or calcic antagonists. The aldosterone antagonist is administered in a daily dose greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take per day. According to this object, the risk of mortality is reduced by a percentage of at least 50%. More preferably, the percent reduction in the risk of mortality ranges between approx. 80% and 50%, 80% and 55%, 80 and 60%, 80% and 65%, 80% and 70%, or between 80% and 75%, and is for instance approx. 80%, 73%, 67%, 65% or 59%. According to this object, the risk of morbi-mortality is reduced by at least 40% or at least 46%.

### BRIEF DESCRIPTION OF FIGURES

FIG. 1: Semi logarithmic graph of the plasmatic concentrations of spironolactone over 48 hours in rat, dog and monkey after administration of 2 mg/kg/day 22-¹⁴C spironolactone marked orally or intravenously (-: total material ¹⁴C; ▴-▴: material ¹⁴C ethyl acetate; ▪-▪: canrenone).

FIG. 2: Semi-logarithmic graph of the time-related concentration of canrenone after administration of doses of spironolactone of 0.8 mg/kg, 2 mg/kg and 8 mg/kg.

FIG. 3: Graph representing the dose/response relation between the dose of spironolactone and the log ratio ([Na⁺]_{urinary}×10/[K⁺]_{urinary}) in 15 dogs in hyperaldosteronemia after a single administration of spironolactone per day. The graph shows that spironolactone enables to restore the Log ratio (Na×10/K) within 0 to 6 hours.

FIG. 4: Graph representing the survival probabilities obtained over 14-15 months, within the framework of clinical studies conducted over a group of dogs having received an oral composition of spironolactone (2 mg/kg/day) and a CEI (spironolactone group) and a second group having received a placebo and a CEI (p=0.011). Spironolactone reduced the risk of mortality by 65%.

FIG. 5: Graph representing the 14-15 month mortality rates, within the framework of clinical studies conducted over a group of dogs having received an oral composition of spironolactone (2 mg/kg/day) and a CEI (spironolactone group) and a second group having received a placebo and a CEI (p=0.0029).

FIG. 6: Graph representing the survival probabilities obtained over 3 years, within the framework of clinical studies conducted over a group of dogs having received an oral composition of spironolactone (2 mg/kg/day) and a CEI (spironolactone group) and a second group having received a placebo and a CEI (p=0.017; Spironolactone reduced the risk of mortality by 59%.

FIG. 7: Graph representing the survival probabilities obtained over 3.5 years, within the framework of clinical studies conducted over a group of dogs treated as of stage I of heart failure and having received an oral composition of spironolactone (2 mg/kg/day) and a CEI (spironolactone group) and a second group having received a placebo and a CEI.

FIG. 8: Graph representing the 14-15 month morbidity-mortality rates, within the framework of clinical studies conducted over a group of dogs having received an oral composition of spironolactone (2 mg/kg/day) and a CEI (spironolactone group) and a second group having received a placebo and a CEI. The morbi-mortality rate is obtained by adding the number of dogs dead, put to sleep or removed from the trial for grounds of worsened heart failure.

FIG. 9: Graph representing the progression of the average concentration of plasmatic potassium or kaliemia observed in the different groups of dogs treated with an oral a composition of spironolactone (2 mg/kg/day) and a CEI (spironolactone group) in comparison with the groups of dogs having received a placebo and a CEI (p<0.05).

### DETAILED DESCRIPTION

The present invention relates to a new veterinary composition intended for the treatment of non-human mammal subjects affected by heart failure comprising an aldosterone antagonist and a pharmaceutically acceptable vehicle, wherein the efficient therapeutic dose of aldosterone antagonist is greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

The present invention also relates to new veterinary compositions intended for the treatment of non-human mammal subjects affected by heart failure comprising an aldosterone antagonist in combination with a standard therapy for the treatment of heart failure, such as for instance CEIs, angiotensin II AT-1-receptor antagonists (ARA-II or sartans), digitalic drugs, inotropes, inodilators, diuretics, vasodilators, beta blockers and/or calcic antagonists and a pharmaceutically acceptable vehicle.

According to the present invention, the aldosterone antagonists include all agents which are capable of bonding to the aldosterone receptor (also called aldosterone receptor antagonist) and acting thus as a competitive aldosterone antagonist by fixing to the binding site of the mineralocorticoids. By way of examples of aldosterone inhibitors, the compounds of spironolactone type comprising a lactone cycle bound to a steroid nucleus may be mentioned.

The new veterinary compositions are hence intended for the treatment of non-human mammal subjects affected by heart failure comprising an aldosterone antagonist receptor, an angiotensin converting enzyme inhibitor (CEI) and a pharmaceutically acceptable vehicle. The compositions in question include an efficient therapeutic dose of aldosterone receptor antagonist of approx. 0.88 to 5 mg/kg/day, and preferably of approximately 2 mg/kg/day. These include besides an efficient therapeutic dose of CEI of approx. 0.1 to 0.6 mg/kg/day, and preferably of approx. 0.25 mg/kg/day. According to the invention efficient therapeutically quantities of a aldosterone receptor antagonist and of an angiotensin converting enzyme inhibitor are used, in view of preparing a veterinary medication intended for reducing the rates of mortality and/or of morbidity of non-human mammal animals affected by heart failure, characterised in that the efficient therapeutic dose of aldosterone receptor antagonist ranges between approx. 0.88 and 5 mg/kg/day, and preferably of approx. 2 mg/kg/day. The method for reducing the rates of mortality and/or of morbidity of non-human mammal animals affected by heart failure includes the administration of efficient therapeutic dose of an aldosterone receptor antagonist and of an angiotensin converting enzyme inhibitor, the aldosterone receptor antagonist being administered in a daily dose ranging between approx. 0.88 and 5 mg/kg/day, and preferably approx. 2 mg/kg/day.

The therapeutic efficiency of the present invention may be expressed as mortality rates observed but also as a risk of mortality. Thus, in the present invention the risk of mortality is reduced by a percentage of at least 50% and the mortality rate of at most 34%. More preferably, the percent reduction in the risk of mortality ranges between approx. 80% and 50%, 80% and 55%, 80% and 60%, 80% and 65%, 80% and 70%, or between 80% and 75%, and is for instance approx. 80%, 73%, 67%, 65% or 59%. Similarly, the most preferable mortality rates obtained are comprised between approx. 34% and 0%, and are for instance approx. 34%, 30%, 20%, 15%, 9%, 6% or 0%. According to this object, the risk of morbi-mortality is reduced by at least 40% or at least 46% to 50%.

The competitive aldosterone antagonists or inhibitors (capable of binding competitively to the aldosterone receptor as described above) may comply with

(i) the following general formula I:

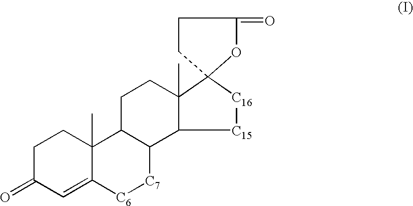

wherein

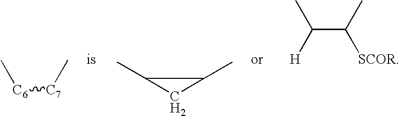

wherein R represents a lower alkyl having 1 to 5 carbon atoms, and wherein

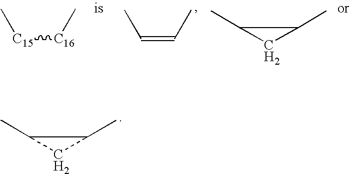

The lower alkyl residues may be linear or not, preferably methyl groups, an ethyl and an n-propyl. Examples of spironolactone type compounds belonging to formula I are listed below. The methods of production of these compounds are well-known in the art and are besides described in U.S. Pat. No. 4,129,564.
7α-Acetylthio-3-oxo-4,15-androstadiene-[17(β-1′)-spiro-5′]perhydrofuran-2′-one;
3-Oxo-7α-propionylthio-4,15-androstadiene-[17((β-1′)-spiro 5′]perhydrofuran-2′-one;
6β,7β-Methylene-3-oxo4,15-androstadiene-[17((β-1′)-spiro-5′]perhydrofuran-2′-one;
15α,16α-Methylene-3-oxo-4,7α-propionylthio-4androstene[17(β-1′)-spiro-5′]perhydrofuran-2′-one;
6β,7β,15α,16α-Diethylene-3-oxo-4-androstene-[17(β-1′)-spiro-5′]perhydrofuran-2′-one;
7α-Acetylthio-15β,16β-Methylene-3-oxo-4-androstene-[17(β-1′)-spiro-5′]perhydrofuran-2′-one;
15β,16β-Methylene-3-oxo-7ss-propionylthio-4-androstene-[17(β-1′)-spiro-5′]perhydrofuran-2′-one;
6β,7β,15β,16β-Dimethylene-3-oxo-4-androstene-[17(β1′)-spiro-5′]perhydrofuran-2′-one; an
10,13-dimethylspiro[2,8,9,11,12,14,15,16-octahydro-1H-cyclopenta[α]phenanthrene-17,5′-oxolane]-2′,3-dione (canrenone)

(ii) the following general formula II:

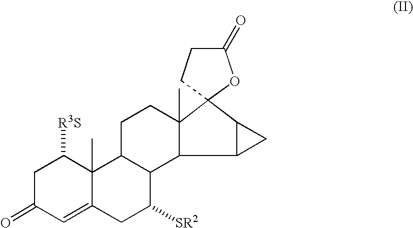

wherein the radical R1 is an alkyl or a C1-C3 acyl and R2 is hydrogen or a C1-C3 alkyl. Compounds belonging to this family are for instance: 1α-acetylthio-15β, 16β-methylene-7α-methylthio-3-oxo-17α-pregn-4-ene-21,17-carbolactone; and 15β, 16β-methylene-1α,7α-dimethylthio-3-oxo-17α-pregn-4-ene-21,17-carbolactone.

(iii) the following general formula III:

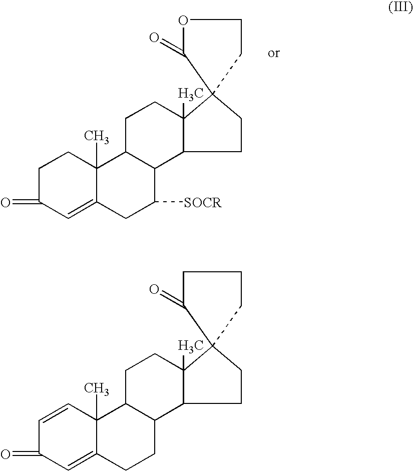

wherein R is a lower alkyl, preferably a methyl, ethyl, propyl and butyl moiety. By way of examples the following may be mentioned:
3β,21-dihydroxy-17α-pregna-5,15-diene-17 carboxylic y-lactone acid;
3β,21-dihydroxy-17α-pregna-5,15-diene-17-carboxylic lactone 3-acetate acid;
3β,21-dihydroxy-17α-pregn-5-ene-17-carboxylic γ-lactone acid;
3β,21-dihydroxy-17α-pregn-5-ene-17-carboxylic γ-lactone 3-acetate acid;
21-hydroxy-3-oxo-17α-pregn-4-ene-17-carboxylic γ-lactone acid;
21-hydroxy-3-oxo-17α-pregna-4,6-diene-17-carboxylic γ-lactone acid;
21-hydroxy-3-oxo-17α-pregna-1,4-diene-17 carboxylic γ-lactone acid;
7α-acylthio-21-hydroxy-3-oxo-17α-pregn-4-ene-17-carboxylique γ-lactone acid; and
7α-acetylthio-21-hydroxy-3-oxo-17α-pregn-4-ene-17-carboxylique γ-lactone acid.

(iv) the following general formula IV:

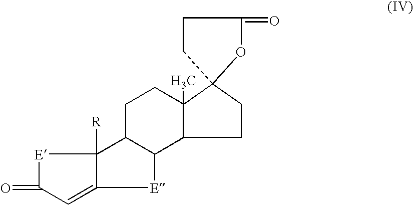

wherein E′ is a moiety selected among ethylene, vinylene and lower alkanoyl thioethylene radicals;
wherein E″ is a moiety selected among ethylene, vinylene, and lower alkanoyl thioethylene radicals, and R is a methyl radical, except when E′ and E″ are ethylene radicals.

(v) the following general formula V, among which for instance, 1-acetylthio-17α-(2-carboxyethyl)-17β-hydroxy-androst-4-en-3-one lactone may be mentioned.

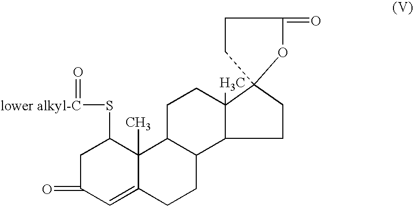

(vi) the following general formula VI. By way of examples the following may be mentioned: 7α-acetylthio-17α-(2-carboxyethyl)-17β-hydroxy-androst-4-en-3-one lactone; 7α-acetylthio-17α-(2-carboxyethyl)-17β-hydroxy-androst-4-en-3-one lactone; 1α,7α-diacetylthio-17α-(2-carboxyethyl)-17ss-hydroxy-androsta-4,6-dien-3-one lactone; 7α-acetylthio-17α-(2-carboxyethyl)-17β-hydroxy-androsta-1,4-dien-3-one lactone; 7α-acetylthio-17α-(2-carboxyethyl)-17β-hydroxy-19-norandrost-4-en-3-one lactone; and 7α-acetylthio-17α-(2-carboxyethyl)-17β-hydroxy-6α-methylandrost-4-en-3-one lactone.

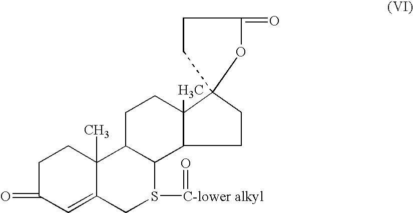

(vii) the following general formula VII:

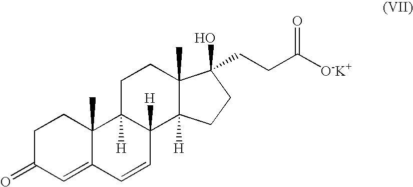

By way of example, potassium 3-[(8R,9S,10R,13S,14S,17R)-17-hydroxy-10,13-dimethyl-3-oxo-2,8,9,11,12,14,15,16-octahydro-1H-cyclopenta[α]phenanthren-17-yl]propanoate (caneroate) may be mentioned

The veterinary compositions according to the present invention include preferably spironolactone, or eplerenone as an aldosterone antagonist. Throughout the present description, the terms spironolactone and eplerenone also contain the derivatives or the metabolites of these compounds. Preferably, the spironolactone used according to the present invention is a 17-lactone synthetic steroid compound belonging to the family (I) described above. More preferably still, 7α-acetylthio-3-oxo-17α-pregn-4-ene-21,17-carbolactone is used. Spironolactone is well-known in human medicine, and it is marketed under the Aldactone®, NovoSpiroton®, Spiractin®, Spirotone®, and Berlactone® trademarks. The general chemical formula is as follows (http://www.chemblink.com):

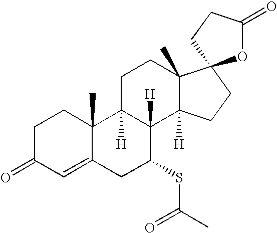

Eplerenone, also designated as epoxymexrenone, is an epoxy derivative: 9,11-epoxy-spirolactone (U.S. Pat. No. 4,559,332). The complete chemical name is pregn-4-ene-7,21-dicarboxylic acid, 9,11-epoxy-17-hydroxy-3-oxo, γ-lactone, methyl ester (7α, 11α, 17α). It is marketed, in human medicine, among others under the Inspra® trademark. The general formula is as follows (http://www.chemblink.com):

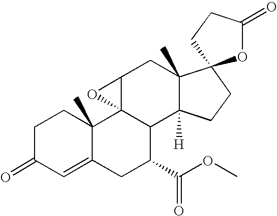

According to the invention, by spironolactone is meant the compound as such, its derivatives and/or its metabolites. By way of examples of spironolactone derivatives, the optically active isomers of spironolactone, the mono or bis-cyclopropyl derivatives of spironolactone or the epoxy derivatives, such as 9α,11α-epoxy spironolactone or eplerenone, or more generally all the functionalised derivatives of active spironolactone may be mentioned, i.e. presenting the therapeutic activity of spironolactone according to the present invention. By way of examples of spironolactone metabolites, canrenone, canrenoic acid, 15β-OH canrenone, 21-OH canrenone, potassium canrenoate, 7α-thio-spironolactone, 7α-thiomethyl-spironolactone or 6-β-hydroxy-7-α-thiomethyl spironolactone may be mentioned without restriction.

The compounds of spironolactone type, such as spironolactone, eplerenone, the derivatives or metabolites of these compounds are administered in larger doses than those used conventionally for treating heart failure in human. These doses are generally qualified as diuretic doses and hyperkaliemia side effects in human patient. It has been discovered according to the present invention that the doses of the compounds of spironolactone type with a diuretic effect in non-human mammal animals which are identical to those causing a diuretic effect in human do not induce, contrary to what is observed in human, hyperkaliemia side effects in the sub-group of patients constituted of non-human mammal animals.

The compositions according to the present invention which include preferably spironolactone or eplerenone or the derivatives, the metabolites of these compounds as an aldosterone antagonist are particularly useful when they are administered according to the posologies prescribed for the treatment and/or the prevention of heart failure in non-human animals such as dog and/or cat, in particular in the early stages of the pathology.

Preferably, the compositions according to the present invention are administered before the onset of edemas in these sick animals.

In human the doses of spironolactone used conventionally are approx. 1 to 25 mg/day with an average daily administration of 12.5 mg/day, but never more than 50 mg/day so as not to cause hyperkaliemia in human. The human doses are sometimes transposed to animals by allometric extrapolation, a method taking different physiological parameters into account such as in particular pharmacokinetics. The allometric equation applied currently is as follows:

Log(Cl)=0.5408×Log(BW)−0.2764

BW: Body Weight

Consequently, the clearance (Cl) or plasmatic purification coefficient of a substance, i.e. the capacity of an member to eliminate totally a given substance of a given volume of arterial plasma per time unit is deduced from the equation: Cl=0.5291×BW^{0,0.408}.

By using this allometric equation the clearances for dogs and men are calculated, with a body weight of 70 kg and of 10 kg respectively for human and dog. The clearances thus calculated are given in Table 1 below.

TABLE 1

| Species | Weight (kg) | Clearance (L/h) |
|---|---|---|
| Dog | 10 | 1.831 |
| Human | 70 | 5.253 |

Consequently, the doses of 12.5 mg; 25 mg; 50 mg and 75 mg (dose in mg/day for a human patient) extrapolated to a dog (dose in mg/kg/day), calculated according to the following equation: Dose_{dog}=(Doseₕᵤₘₐₙ×Cl_{dog})/Clₕᵤₘₐₙ, are presented in table 2 below:

TABLE 2

| Doses in mg/day | Equivalent in mg/kg/day | |
|---|---|---|
| (human patient) | (non-human mammal animals) | Therapeutic effect in human |
| 12.5 mg/day | 0.436 mg/kg/day | Significant reduction in morbidity/ |
| | | mortality, without hyperkaliemia |
| 25 mg/day | 0.871 mg/kg/day | Significant reduction in morbidity/ |
| | | mortality, without hyperkaliemia |
| 50 mg/day | 1.743 mg/kg/day | Significant reduction in morbidity/ |
| | | mortality and onset of a (slight) diuretic |
| | | effect and a (moderate) hyperkaliemia |
| 75 mg/day | 2.614 mg/kg/day | Significant reduction in morbidity/ |
| | | mortality and onset of a (moderate to |
| | | strong) diuretic effect and a (moderate to |
| | | severe) hyperkaliemia |

According to the present invention, the efficient therapeutic proportions of aldosterone antagonist, without hyperkaliemia side effects in non-human mammal animals, are greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

Indeed, the therapeutically efficient proportions without transient side effects in non-human mammal animals of aldosterone receptor antagonist are approx. 0.88 to 5 mg/kg/day, or approximately 1 to 5 mg/kg/day, or approximately 1 to 4 mg/kg/day, or still approx. 1 to 3 mg/kg/day and preferably of approx. 2 mg/kg/day or approx. 4 mg/kg/day.

Such doses of spironolactone are generally established as also having a diuretic effect in human, and cannot hence be used in human on their own or in combination with CEIs, since they cause a strong hyperkaliemia side effect in human patients thus treated, which is not compatible with the desired treatment of heart failure.

It has been noticed surprisingly that such high doses of spironolactone greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day and administered every 24 hours in a single take, do not induce similar hyperkaliemia side effects in non-human mammal animals or only induce small and transient hyperkaliemia. Indeed, when the non-human mammal animals are treated with the compositions according to the present invention, the circulating potassium rates remain substantially constant or are little and transitorily increased to concentrations ranging between 5.9 to 6.4 mmol/L or ranging between 6.5 and 7.5 mmol/L, and remain smaller than 7.5 mmol/L. No significant hyperkaliemia side effect has been observed when treating non-human mammal animals as demonstrated besides in the examples.

The doses of aldosterone antagonist, for instance spironolactone, eplerenone, of the derivatives or metabolites of these compounds are suited to each of the mammals treated according to the weight and in order to comply with the posology prescribed by the present invention greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

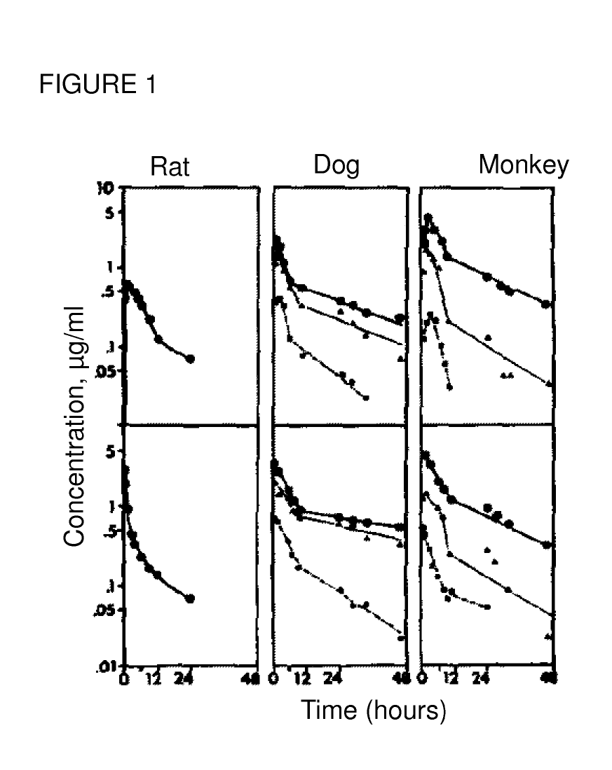

According to the present invention, a single administration per 24 hours of aldosterone antagonist such as spironolactone at high dose is performed, whereas 1a plasmatic concentration of spironolactone decreases rapidly 4 hours after oral administration (FIG. 1). The aldosterone antagonists such as spironolactone, its derivatives, or its metabolites, are preferably administered to sick animals, in a single take once a day, for instance when eating, either mixed to the food ration, or directly into the mouth after the meal. Preferably, the compositions according to the present invention are administered in the early stages of heart failure and in particular before the onset of edemas in these sick animals. No hyperkaliemia side effect could be observed in the animals thus treated.

As demonstrated in particular in Example 1 below, the optimal daily dose of spironolactone has been determined in non-human animals such as dog for instance. This dose is surprisingly vastly greater than the dose used currently for treating human patients. This optimal dose is greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day, and may reach 6 or 7 mg/kg/day for treating non-human animals, with maximum 8 mg/kg/day.

The use of these new posologies of spironolactone enables to restore the sodium/plasmatic potassium ratio induced by hyperaldosteronemia reproducing the model of heart failure. The use of these new posologies of spironolactone enables to restore sodium and potassium physiological urinary concentrations and to normalise the ratio ([Na⁺]_{urinary}×10/[K⁺]_{urinary}). The therapeutically efficient and non-toxic doses of spironolactone, its derivatives, or its metabolites according to the present invention are greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day and administered every 24 hours in a single take to animals in order to normalise the ratio log([Na⁺]_{urinary}×10/[K⁺]_{urinary}) and to treat and/or prevent the major pathologies in non-human animals, affected by heart failure.

According to the present invention, the compositions of spironolactone, its derivatives, or its metabolites, administered in a single take and in a dose greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day are particularly useful in order to process non-human animals affected by heart failure, without hyperkaliemia side effects. Heart failure originates from cardiopathies which may fall into two categories: congenital cardiopathies or acquired cardiopathies. The former are congenital cardiac malformations. Contrary to congenital affections, acquired cardiopathies appear during the life of the animal, generally at a later stage (>6-8 years). They are of various origins, but two affections predominate quite clearly in dogs: Degenerative Valvular Disease (DVD) and dilated cardiomyopathy (DCM).

DVD which is also called valvular endocardiosis, valvular failure or valvulopathy, represents 80% of cardiopathies in dogs. DVD is characterised by an alteration in the atrioventricular valves (mainly mitral, sometimes mixed) causing poor impermeability during ventricular systole. Blood is then regurgitated into the atrium which the volume of systolic ejection to drop and an overload in the atrium. When valvular lesions progress, the tendinous cords may even be attacked and fractured, thereby causing valvular leak and endangering the vital prognosis. The first way of detecting DVD is auscultation; the mitral leak brings about a murmur (left apexian systolic), whereof the intensity is correlated to the magnitude of the regurgitation, echocardiography is also vastly used. As the affection progresses, an atrial dilatation can first be observed, then a ventricular dilatation. At this stage, the systolic function is altered very early. Once the mitral valve is hit, the resulting heart failure is first of all on the left; in advanced stages, it may become global, left and right-sided. Setting up compensatory mechanisms is done gradually, the onset of Congestive Heart Failure (CHF) is relatively late and induced pulmonary edemas and hypertension during left-sided CHF; ascites during right-sided heart failure; DVD evolves over months or years.

DCM is a primitive myocard affection in non-human animals, dogs or cats. In its conventional form, it is shown by thinning walls in the ventricular myocard and dilated heart cavities. The systolic function is attacked early and severely. DCM is an affection evolving generally quite rapidly, with the onset of sudden and decompensated CHF.

The compositions of spironolactone, its derivatives or its metabolites as described previously also enable to prevent and/or to treat pathologies in cats like feline hypertrophic cardiomyopathies (DCM, or dilated cardiomyopathies and/or HCM, or hypertrophic cardiomyopathies). The latter are characterised by a thickening of the ventricular myocard which gradually reduces the volume of the ventricular cavity. The volume of blood that the cavity may intake is thereby reduced, which eventually induces, as in the case of DCM, congestive heart failure (CHF).

Preferably according to the present invention, the compositions of spironolactone, its derivatives or its metabolites, and dosages described previously are particularly useful for treating non-human mammal animals affected by congestive heart failure with valvular regurgitation. As described previously, the cardiac valves then fail and the heart cannot perfuse the different organs sufficiently any longer. Blood stagnates in the veins, and plasmatic liquid diffuses through the tissues, causing edemas and effusions.

By non-human mammal animals is meant generally all the species of mammals. Preferably, the compositions according to the present invention are intended for pets, such as for instance dogs, cats and horses.

The compositions comprising high doses of aldosterone antagonist, such as spironolactone, its derivatives or its metabolites are used in combination with a standard therapy for the treatment of heart failure. Preferably, the compositions of spironolactone, its derivatives or its metabolites are used in combination with a standard therapy for the treatment of congestive heart failure.

According to the invention, by standard heart failure therapy is meant CEIs, angiotensin II AT-1-receptor antagonists (ARA-II or sartans), digitalic drugs, inotropes, inodilators, diuretics, vasodilators, beta blockers and/or calcic antagonists.

According to a preferred embodiment, the compositions of spironolactone, its derivatives or its metabolites according to the present invention are used in combination with a CEI. Among CIEs, Benazepril, Enalapril, Captopril, Cilazapril, Fosinopril, Imidapril, Lisinopril, Moexipril, Perindopril, Quinapril, Ramipril, Spirapril or Trandolapril still, may be mentioned in particular. Preferably, Benazepril or Enalapril is used in the compositions according to the present invention in combination with spironolactone, its derivatives or its metabolites. The efficient therapeutic doses of angiotensin converting enzyme inhibitor used in the compositions are approx. 0.1 to 0.6 mg/kg/day, preferably approximately 0.25 mg/kg/day for Benazepril and 0.5 mg/kg/day for Enalapril.

Alternately, the compositions of spironolactone, its derivatives or its metabolites according to the present invention are used in combination with angiotensin II AT-1-receptor antagonists, also designated ARAII or sartans. These compounds act as competitive inhibitors of angiotensin II near the AT-1 receptor, thereby blocking the effect of angiotensin II near the AT-1 receptor of angiotensin. By way of examples of these compounds, candesartan, candesartan cilexetil, prosartan, irbesartan, losartan, losartan potassic salt, olmesartan, telmisartan or valsartan may be mentioned. These are used in the associations according to the present invention in efficient therapeutic doses.

According to another preferred embodiment of the present invention, the compositions include efficient therapeutic quantities of aldosterone antagonists in a single daily take, such as spironolactone, its derivatives or its metabolites according to the posologies prescribed previously in combination with an efficient therapeutic quantity of inotrope or inodilator such as for instance pimobendane or levosimendane. Pimobendane corresponds to 4,5-dihydro-6-[2-(4-methoxyphenyl)-1H-benzimidazol-5-yl]-5-methyl-3 (2H)-pyridazone whereof the chemical structure is as follows:

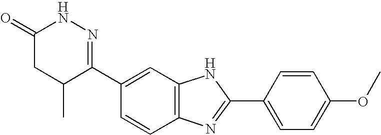

Pimobendane is described among others in U.S. Pat. No. 4,361,563 and EP008391 and is marketed under the Vetmedin® name by Boehringer Ingelheim. By its action mechanisms (calcium sensitizer and inhibitor of phosphodiesterase III), it is a positive inotrope (increased contractility), positive lusitrope (improved relaxation) and arterial (reduced post-charge), venous (reduced pre-charge) and coronary (improved myocard oxygen delivery) vasodilator. Its positive inotrope action (linked with increased affinity of troponin for calcium) is exerted without increase in myocardial energy consumption. The vasodilating effect is intense and direct (by inhibiting degradation of AMPc in the smooth muscle cell of vessels). Efficient therapeutic doses of pimobendane administered orally with the compositions according to the invention. For instance, these doses may be approx. 0.25 to 2 mg/kg/day and preferably approx. 0.5 mg/kg/day.

Levosimendane which is describes as an inodilator in the European patent EP383449 may also be mentioned, and corresponds to [[4-(1,4,5,6,-tetrahydro-4-methyl-6-oxo-3-pyridazinyl)phenyl]hydrazono]propane nitrile where of the chemical structure as follows:

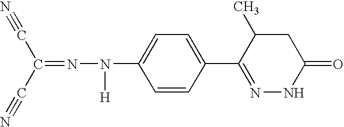

Levosimendane can be administered orally or by injection. Efficient therapeutic doses of levosimendane used in the compositions according to the present invention. These doses may be for instance ranging between 0.025 and 0.5 mg/kg/day according to the way of administration selected. By way of examples when they are administered orally, the efficient therapeutic doses may be 0.1 mg/kg to 0.2 mg/kg in two takes (0.05 to 0.1 mg/kg morning and evening).

More preferably, the compositions include efficient therapeutic quantities of aldosterone antagonists, such as spironolactone, its derivatives or its metabolites according to the posologies prescribed previously in combination with efficient therapeutic quantities of inotropes or inodilators such as for instance pimobendane or levosimendane and CEI; such as benazepril or enalapril. these compositions are particularly efficient for treating and/or preventing DVDs in dogs, DCMs in dogs and cats as well as HCM in cats.

The aldosterone antagonist compositions such as spironolactone, its derivatives, or its metabolites according to the invention can be administered with vasodilators, such as nitroprusside sodium, nitroglycerin or isosorbide nitrate, and/or diuretics such as furosemide, bumetanide, torasemide or thiazidic drugs such as chlorothiazide or hydrochlorothiazide. Diuretics are used in case of marked congestive signs (pulmonary edema, ascite . . . ) in the smallest dose necessary. When the compositions according to the invention are administered in combination with a diuretic, the latter is preferably furosemide in a 4-8 mg/kg/day dose and is also administered.

The aldosterone antagonist compositions such as spironolactone, its derivatives or its metabolites described previously can moreover be administered with other usual treatments for heart failure such as digitalic drugs, for example digoxin, as a standard therapy for heart failure in particular for treating supraventricular artyhmias, in particular atrial fibrillation.

The daily doses of these usual treatments for heart failure are adapted to each of the non-human mammals affected by heart failure, and treated with the compositions according to the present invention.

The compositions are useful for treating and/or prevent degenerative valvular diseases (DVD) in dogs, dilated cardiomyopathy (DCM) in dogs and cats as well as hypertrophic cardiomyopathy (HCM) in cats. Also, they are particularly appropriate for treating non-human animals affected by heart failure.

By efficient or active therapeutic dose is meant a quantity capable of restoring the sodium/plasmatic potassium ratio and/or of inducing sufficient therapeutic effect and thus provide a significant reduction in the mortality rate and/or the morbidity rate. Also, by active therapeutic dose is meant a quantity of each of the active ingredients capable of causing in combination a sufficient therapeutic effect and thus a reduction in mortality and/or morbidity.

According to the present invention, the risk of mortality observed is reduced by a percentage of at least 50%. More preferably, the percent reduction in the risk of mortality ranges between approx. 80% and 50%, 80% and 55%, 80% and 60%, 80% and 65%, 80% and 70%, or between 80% and 75%, and is for instance approx. 80%, 73%, 67%, 65% or 59%. The protective effect is particularly high for animals affected by heart failure including the early stages. The risk of morbi-mortality is reduced by at least 40% or at least 46%.

The compositions of spironolactone, its derivatives or its metabolites in single administration per 24 h and/or efficient therapeutic quantities of CEI, angiotensin II AT-1-receptor antagonists (ARA-II or sartans), digitalic drugs, inotropes, inodilators, diuretics, vasodilators, beta blockers and/or calcic antagonists are efficient for the treatment and/or prevention of heart failures in non-human mammal animals. The compositions according to the present invention are particularly efficient for treating congestive heart failures with valvular regurgitation. Indeed, it has been demonstrated that dogs suffering in particular from congestive heart failure due to valvular regurgitation to which the compositions of spironolactone, its derivatives or its metabolites according to the posologies described previously are administered in a single take in combination with a standard therapy, show a longer lifetime than dogs having received a standard therapy on its own. This posology may be maintained in long-term treatment for instance over 15 months or 36 months. Indeed, in the long term, it has been demonstrated that heart failure in non-human animal subjects has less worsened than in dogs having received a standard therapy only.

Non-human mammal animals may receive preferably therapeutically efficient doses of spironolactone and benazepril. These can be administered in sequence or simultaneously according any well-known ways of administration in the art and suited to the treatment of each animal, for example nasal, oral and parenteral. The methods according to the invention enable to treat subjects affected by heart failure, in particular pets such as for instance dogs, cats or horses. When the methods according to this embodiment are intended for the treatment of dogs affected by heart failure, a daily dose of aldosterone receptor antagonist ranging between 0.88 and 5 mg/kg/day and preferably approximately 2 mg/kg/day, and a daily dose of angiotensin converting enzyme inhibitor ranging between 0.1 and 0.6 mg/kg/day and preferably approx. 0.25 mg/kg/day are administered. When the methods according to this embodiment are intended for the treatment of cats affected by heart failure, a daily dose of aldosterone receptor antagonist ranging between 0.88 and 5 mg/kg/day, and preferably approximately 2 mg/kg/day, and a daily dose of angiotensin converting enzyme inhibitor ranging between 0.1 and 0.6 mg/kg/day and preferably approx. 0.25 mg/kg/day are then administered. Finally, when the methods according to this embodiment are intended for the treatment of horses affected by heart failure, a daily dose of aldosterone receptor antagonist ranging between 0.88 and 5 mg/kg/day, and preferably approximately 2 mg/kg/day, and a daily dose of angiotensin converting enzyme inhibitor ranging between 0.1 and 0.6 mg/kg/day and preferably approx. 0.25 mg/kg/day are administered. As indicated previously, the administrations can be simultaneous or in sequence.

The veterinary compositions or medications according to the present invention can be in any appropriate forms to suit the requested administration modes, for instance nasal, oral, intradermic, cutaneous or parenteral. They may hence be in the form of a nasal, oral or injectable liquid suspension or solution, or in solid or semi-solid form, powders, pellets, capsules, granules, sugar-coated pills, gelules, sprays, cachets, pills, tablets, pastes, implants or gels.

According to the formulations of the compositions and medications used, they may include moreover ingredients used conventionally in pharmacy for the preparation of liquid or solid formulations for nasal, oral, intradermic, cutaneous or parenteral administration. Thus the compositions according to the invention may include according to the type of formulations, a flow agent, a lubricant and any excipient of convenient mass, such as lactose, cellulose or starches. As a lubricant, stearic acid, magnesium stearate, L-leucine or for instance, glycerol tribehenate. As a disintegration agent, sodic carboxymethylamidone, cross-linked sodic carboxymethylcellulose or, for instance, cross-linked polyvinylpyrrolidone may be used. As a flow agent, pure silica or colloidal silicon dioxide may be used.

The oral forms of medication may be instant dissolution pellets or effervescent obtained by adding an effervescent couple to the composition according to the invention, or still coated pellets. As an effervescent couple, tartaric acid and sodium bicarbonate or citric acid and sodium bicarbonate may be used.

When the compositions are in the form of pellets, they are for instance 10 mg, 40 mg or 80 mg spironolactone pellets. The pellets are divisible so that they can be cut to suit the posology according to the invention in a single daily take.

The injectable preparations are produced by mixing therapeutically efficient quantities of aldosterone antagonists and for instance CEI and/or an inotrope, possibly an inodilator, with a pH regulator, a buffer agent, a suspension agent, a solubilisation agent, a stabilizer, a tonicity agent and/or a preservative, and by transformation of the mixture into an intravenous, sub-cutaneous, intramuscular injection or perfusion, according to a conventional method. Possibly, the injectable preparations may be lyophilised according to a conventional method. Examples of suspension agents include methylcellulose, polysorbate 80, hydroxyethylcellulose, xanthan gum, sodic carboxymethylcellulose and polyethoxylated sorbitan monolaurate. Examples of solubilisation agent include polyoxyethylene-solidified castor oil, polysorbate 80, nicotinamide, polyethoxylated sorbitan monolaurate, macrogol and ethyl ester of caste oil fatty acid. Moreover, the stabilizer includes sodium sulfite, sodium metalsulfite and ether, while the preservative includes methyl p-hydroxybenzoate, ethyl p-hydroxybenzoate, sorbic acid, phenol, cresol and chlorocresol. An example of tonicity agent is mannitol. When preparing injectable suspensions or solutions, it is desirable to make sure that they are blood isotonic.

The present invention also relates to a kit for veterinary usage intended for the treatment of non-human mammal subjects affected by heart failure, having at least one compartment for a sterile packaging or not, separate or not, for simultaneous or sequential administration of daily doses of aldosterone antagonist only or in association with a standard heart failure therapy, such as for instance a CEI, an angiotensin II AT-1-receptor antagonist (ARA-II or sartans), a digitalic drug, an inotrope, an inodilator, a diuretic, a vasodilator, a beta blockers and/or a calcic antagonist. The compartment(s) may thus contain a daily dose of aldosterone antagonist greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day, and a daily dose of an angiotensin converting enzyme inhibitor of approx. 0.1 to 0.6 mg/kg/day, preferably approximately 0.25 mg/kg/day for Benazepril and 0.5 mg/kg/day for Enalapril, and/or approx. 0.25-2 mg/kg/day Pimobendane, and/or approx. 0.025-0.5 mg/kg/day Levosimendane.

The kit according to this embodiment facilitates administration of the posologies prescribed for each subject, simultaneously or in sequence, and at least once a day. Also, the kit according to the invention includes a sterile or non-sterile packaging of the aldosterone antagonist with or without standard therapy, adapted for oral, nasal, intradermic, cutaneous, or parenteral administration, as well as the means enabling these formulations to be administered. Finally, the kits according to the present invention include moreover a location for an instruction sheet regarding the operating mode and the administration mode of said formulations.

The aldosterone antagonists and the standard treatments of heart failure, such as CEIs, angiotensin II AT-1-receptor antagonists (ARA-II or sartans), digitalic drugs, inotropes, inodilators, diuretics, vasodilators, beta blockers and/or calcic antagonists are such as described previously for the compositions and may advantageously be delivered simultaneously or in sequence, by nasal, oral, intradermic, cutaneous or parenteral administration. Also, the doses appropriate for each of the non-human mammal animals to be treated are as described previously for instance for dogs, cats or horses.

The present invention moreover relates to the use of efficient therapeutic quantities of an aldosterone antagonist such as spironolactone, its derivatives, or its metabolites in view of the preparation of a veterinary medication for the prevention and/or the treatment of non-human animals affected by non-decompensated heart failure, without causing any hyperkaliemia side effects, wherein said aldosterone antagonist is administered in a daily dose greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take. Preferably, the compositions according to the present invention are administered in the early stages of heart failure and in particular before the onset of edemas in non-human animals thus treated.

Preferably, the present invention moreover relates to the use of efficient therapeutic quantities of an aldosterone antagonist in combination with a standard therapy for heart failure, such as CEIs, angiotensin II AT-1-receptor antagonists (ARA-II or sartans), digitalic drugs, inotropes, inodilators, diuretics, vasodilators, beta blockers and/or calcic antagonists, in view of preparing a veterinary medication intended for reducing the rates of mortality and/or of morbidity of non-human mammal animals affected by heart failure, without causing any hyperkaliemia side effects, wherein said aldosterone antagonist is administered in a daily dose greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take.

The aldosterone antagonists are as described previously and are preferably selected among spironolactone or eplerenone or derivatives, metabolites of these compounds. The standard therapies of heart failure are as described previously and may be selected among CEIs, angiotensin II AT-1-receptor antagonists (ARA-II or sartans), digitalic drugs, inotropes, inodilators, diuretics, vasodilators, beta blockers and/or calcic antagonists. CEIs are for instance alacepril, benazepril, captopril, cilazapril, delapril, enalapril, enalaprilat, fosinopril, fosinoprilat, imidapril, idrapril, lisinopril, perindopril, quinapril, ramipril, saralasin acetate, perindropilat, temocapril, trandolapril, ceranapril, moexipril, quinaprilat, spirapril, a salt or a pharmaceutically acceptable ester of these compounds. Preferably, benazepril, its derivatives such as benazepril chlorhydrate and/or enalapril are used.

The uses according to this embodiment enable to prepare medications for veterinary usage for treating non-human mammal animals, in particular pets, such as for instance dogs, cats or horses. These uses enable in particular the preparation of medications for veterinary usage for treating DVDs in dogs, DCMs in dogs and cats, and HCMs in cats.

When the veterinary medications are used for treating dogs, the daily dose of aldosterone antagonist is greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take. The daily dose of angiotensin converting enzyme inhibitor ranges between 0.1 and 0.6 mg/kg/day and preferably approx. 0.25 mg/kg/day for benazepril and 0.5 mg/kg/day for enalapril. These may also include an efficient therapeutic dose of an inotrope such as pimobendane or levosimendane. For instance, an efficient therapeutic dose may range between 0.25-2 mg/kg/day for pimobendane and 0.025-0.5 mg/kg/day for levisomendane.

As described previously, the veterinary compositions and the medications intended for treating pets may be in any appropriate forms to suit the requested administration modes, for instance nasal, oral, intradermic, cutaneous or parenteral. They may hence be in the form of an oral or injectable liquid solution, or in the form of a suspension or in solid or semi-solid form, powders, pellets, capsules, granules, sugar-coated pills, gelules, sprays, cachets, pills, tablets, pastes, implants or gels.

Another object still of the present invention consists of a method for treating and/or preventing non-human animals affected by non-decompensated heart failure, without causing any non-reversible hyperkaliemia side effects, comprising the administration of aldosterone antagonist compositions, such as for instance spironolactone, its derivatives or its metabolites, in a single per day take, and according to the posologies described above. The treatment methods according to the present invention consist preferably in administering said compositions in the early stages of heart failure, in particular before the onset of edemas in non-human animals thus treated.

The present invention also relates to a method for treating non-human animals affected by heart failure, in particular DVDs in dogs, DCM in dogs and cats, and HCM in cats, comprising the administration of aldosterone antagonist compositions, such as for instance spironolactone, its derivatives, or its metabolites, in a single per day take, and according to the posologies of the present invention in combination with a standard heart failure therapy.

The present invention also relates to a method for reducing the rates of mortality and/or of morbidity of the non-human mammal animal subjects affected by heart failure comprising the administration of efficient therapeutic quantities of an aldosterone antagonist solely or in combination with CEIs, angiotensin II AT-1-receptor antagonists (ARA-II or sartans), digitalic drugs, inotropes, inodilators, diuretics, vasodilators, beta blockers and/or calcic antagonists. According to the invention, the aldosterone antagonist is administered in a daily dose greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably of approximately 2 mg/kg/day or 4 mg/kg/day in a single take, and the risk of mortality observed is reduced by at least 50%. More preferably, the reduction in the risk of mortality ranges between approx. 80% and 50%, between 80% and 55%, between 80% and 60%, between 80% and 65%, between 80% and 70%, or between 80 and 75% and is for instance approx. 80%, 73%, 67%, 65% or 59%. The protective effect is particularly high for animals affected by heart failure including the early stages of the pathology. According to this embodiment, the risk of morbi-mortality is reduced by at least 40% or at least 46%. Besides, the treatment methods according to the invention do not cause any hyperkaliemia side effects. According to the treatment methods of the invention, the aldosterone antagonist may be used on its own or in combination with a standard heart failure therapy, which is then administered in a therapeutically efficient dose of approx. 0.1 to 0.6 mg/kg/day, such as preferably approximately 0.25 mg/kg/day for Benazepril and 0.5 mg/kg/day for Enalapril, and/or a dose of 0.25-2 mg/kg/day for Pimobendane and/or approx. 0.025-0.5 mg/kg/day for Levosimendane.

The aldosterone antagonists, as well as the standard heart failure therapies, as described previously may be used in the method of the present invention. Preferably, spironolactone or eplerenone or the derivatives, the metabolites, of these compounds is administered as an aldosterone antagonist according to the treatment methods of the present invention. When spironolactone, its derivatives, CEIs are for instance alacepril, benazepril, captopril, cilazapril, delapril, enalapril, enalaprilat, fosinopril, fosinoprilat, imidapril, idrapril, lisinopril, perindopril, quinapril, ramipril, saralasin acetate, perindropilat, temocapril, trandolapril, ceranapril, moexipril, quinaprilat, spirapril, a salt or a pharmaceutically acceptable ester of these compounds, for instance with CIEs, these may be selected among alacepril, benazepril, captopril, cilazapril, delapril, enalapril, enalaprilat, fosinopril, fosinoprilat, imidapril, idrapril, lisinopril, perindopril, quinapril, ramipril, saralasin acetate, perindropilat, temocapril, trandolapril, ceranapril, moexipril, quinaprilat, spirapril, a salt or the pharmaceutically acceptable esters of these compounds.

Non-human mammal animals may receive preferably therapeutically efficient doses of spironolactone, its derivatives, or its metabolites and benazepril or enalapril and/or pimobendane and/or levosimendane. These can be administered in sequence or simultaneously according any well-known ways of administration in the art and suited to the treatment of each animal, such as nasal, oral, intradermic, cutaneous and parenteral.

The methods according to the invention enable to treat subjects affected by heart failure, in particular pets such as for instance dogs, cats or horses.

When the methods according to this embodiment are intended for the treatment of dogs affected by DVD or DCM, a daily dose of aldosterone antagonist ranging between 0.88 and 5 mg/kg/day, 1.5 mg/kg/day 1.5-5 mg/kg/day, 1.8-5 mg/kg/day, or 2-5 mg/kg/day, and preferably approximately 2 mg/kg/day in a single take, and in combination with a standard therapy such as CEI in a dose of 0.1 and 0.6 mg/kg/day and/or an inotrope (possibly an inodilator), such as pimobendane. An efficient therapeutic dose of pimobendane may be for instance 0.25-2 mg/kg/day.

When the methods according to this embodiment are intended for the treatment of cats affected by DCM or HCM, a daily dose of aldosterone antagonist greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably approx. 2 mg/kg/day or 4 mg/kg/day in a single take can be administered on its own or in association for instance with a dose of CEI ranging between 0.1 and 0.6 mg/kg/day and/or a dose of pimobendane of 0.25-2 mg/kg/day. Cats affected by HCM are treated, according to the invention by the administration of a daily dose of aldosterone antagonist greater than 1 mg/kg/day and smaller than 5 mg/kg/day, ranging between 1.5 and 5 mg/kg/day, 1.8 and 5 mg/kg/day, 1.5 and 4 mg/kg/day, 1.5 and 3 mg/kg/day, preferably ranging between 2 and 5 mg/kg/day, and even more preferably approx. 2 mg/kg/day or 4 mg/kg/day in a single take can be administered on its own or in association for instance with a dose of CEI ranging between 0.1 and 0.6 mg/kg/day.

These administrations can be performed separately or in association simultaneously or in sequence. Also, according to the methods of the invention, the administered compositions as described previously may be in various forms such as for instance liquid solutions, as suspensions, solid or semi-solid, in the form of powders, pellets, capsules, granules, sugar-coated pills, gelules, sprays, cachets, pills, tablets, pastes, implants or gels.

### EXAMPLES

### Example 1

Pharmacokinetic studies on spironolactone for oral administration have been performed on different species, such as rats, dogs and monkeys by using marked spironolactone (22-¹⁴C spironolactone). The results have been presented in the form of logarithmic curves in FIG. 1, and show high plasma radioactivity percentage in rats (66%) and dogs (76%) and lower in monkeys (33%) after 4 hour oral administration of spironolactone.

It has been discovered according to the present invention that contrary to the doses used for treating human patients, the optimal dose of spironolactone for treating heart failure in pets such as dogs, cats, horses was close to 2 mg/kg/day. The changes in logarithmic values [(Na⁺]_{urinary}×10/[K⁺]_{urinary}) induced by aldosterone have been measured after spironolactone treatment.

To conduct these studies, healthy beagle breed dogs (n=15) less than one year old, and weighing between 11.9 and 14.3 kg at the beginning of the study have been used. They have been tattooed in advance for easy identification and situated in stainless steel individual boxes. The temperature of the room was maintained between 17-21° C. and humidity between 45 and 65%. Besides, the rooms were lit for 12 hours then placed in the dark for 12 hours.

The model for assessing the anti-aldosterone activity as described by Hofman L. M et al. (1975, The Journal of Pharmacology and Experimental Therapeutics. 194, 450-456) was used. According to this experimental model, aldosterone was injected immediately after oral administration of spironolactone pellets (10 mg, 40 mg or 80 mg). A preliminary study was performed in order to determining the ED₈₀ value for the natriuretic effect of aldosterone on its own. All the animals had been dosed successively with a vector (control group) then at hand of aldosterone administered in increasing doses of 0.3 μg/kg; 1 μg/kg and 3 μg/kg. An elimination period of at least 48 hours was left between each administration. The 3 μg/kg optimal dose of aldosterone was thus selected at the end of this preliminary study, and constitutes a reference dose enabling to establish correlation with aldosteronemia observed in dogs suffering from heart failure.

The dogs described above were allocated to different treatment groups (A, B, C, D, and E) and a rest period was left between treatments.

The spironolactone doses tested were force-fed in a single administration. The spironolactone pellets were divided in two parts and a maximum of three pellets was administered to dogs so as to reach the requested dose. A negative control group A only received the pharmaceutical excipient. The positive control group B only received 3 μg/kg of aldosterone. The group C received 3 μg/kg aldosterone and 0.88 mg/kg spironolactone. The group D received 3 μg/kg aldosterone and 2 mg/kg spironolactone. The group E received 3 μg/kg aldosterone and 8 mg/kg spironolactone. The animals had been kept in their individual boxes on an empty stomach and their urines were collected approx. 16 h before treatment. 200 g food was then given immediately after treatment.

Blood samples (5 ml) were taken from the cephalic vein immediately after spironolactone administration, then 3, 6, 9, 12 and 24 hours after treatment. The tubes were centrifuged rapidly at 1500 rpm for 10 minutes (at a temperature of +4° C.±2° C.), and the plasma was distributed in two propylene tubes (1.2 mL). The samples were frozen and kept in the dark at approx. −80° C. On the day of the fest, the bladders of the animals were emptied by catherisation, then 6 and 24 hours after treatment. Also, the urine was collected during the time periods ranging from T₀-T₆ₕ; T₆ₕ-T₁₂ₕ and T₁₂ₕ-T₂₄ₕ

The plasmatic aldosterone concentrations were then measured by solid-phase radioimmunoassay (Coat-A-Count® Aldosterone) based upon the use of an aldosterone-specific antibody immobilised on the wall of the polypropylene tube. ¹²⁵I-aldosterone marked enters competes against the running aldosterone of the sample (200 μL) for bonding to the antibody. After calibration and count, the quantity of aldosterone present in the sample was thus determined once counted. It ranged between 25 and 1200 pg/mL.

The sodium and potassium contents in urines were determined using an osmometer. An HPLC detection method coupled to UV-detection was used for measuring the plasmatic levels of spironolactone and of the metabolites (7α-thiomethyl-spirolactone and canrenone). According to this method 500 μL plasma were mixed with ethyl acetate (80/20, V/V) as solvents. Spironolactone, 1,1,1 trichloroethane 7α-thiomethyl-spirolactone and canrenone were subjected to a liquid-liquid extraction. The compounds and the internal standard (i.e. methyl-testosterone) were separated on a Kromasil C18 column. The quantification levels were 10 μg/L for all compounds within +/−14% variation.

The pharmacokinetic studies of spironolactone and metabolite plasmatic concentrations were conducted using a linear regression analysis programme (Kinetica version 4.0, THERMO ELECTRON Corporation, USA).

The Na⁺/K⁺ response to the administration of spironolactone was assessed by measuring the logarithmic ratio ([Na⁺]_{urinary}×10/[K⁺]_{urinary}) of the Na⁺/K⁺ urinary concentrations collected at instant 0, i.e. just after spironolactone administration and 6 hours after spironolactone administration. No correction was made to account for the basic logarithmic levels of ([Na⁺]_{urinary}×10/[K⁺]_{urinary}), and only the Eₘₐₓ model was carried out after spironolactone administration. The relation between the logarithmic ratio ([Na⁺]_{urinary}×10/[K⁺]_{urinary}) during the first 6 hours while the effect was measured, was analysed in relation to the spironolactone dose according to the conventional sigmoid model Eₘₐₓ Equation 3:

$${E}_{lo g ( {Na}^{+} * 10 / {K}^{+} )} = {E}_{0} + \frac{( {E}_{ma x} - {E}_{0} ) \times {Dose}_{( spironolactone m g / kg )}}{{ED}_{50} \times {Dose}_{( spironolactone m g / kg )}} \qquad Eq . 3$$

wherein E₀ is the basic effect measured as a change in the log ([Na⁺]_{urinary}×10/[K⁺]_{urinary}) during the period from 0 to 6 hours after administration of aldosterone on its own (positive control), Eₘₐₓ is the response maximum of the Na⁺ and K⁺ contents expressed in terms of log ([Na⁺]_{urinary}×10/[K⁺]_{urinary}) during the period from 0 to 6 hours after administration of aldosterone, ED₅₀ corresponds to the quantity of spironolactone necessary to reach 50% of the maximum response (i.e. half_{log([Na+]urinary×10/[K+])max}), Eₘₐₓ−E₀ is the difference in the measured effect of the log(Na⁺*10/K⁺) during the period from 0 to 6 hours after administration of aldosterone, E_{log(Na+*10/K+)} is the effect in the presence of spironolactone. The_{(spironolactone, mg/kg)}, E₀, E_{log([Na+]urinary×10/[K+])max} dose and ED₅₀ were obtained by non-linear regression, and n is the Hill coefficient describing the dose-effect relation.

A statistical analysis was performed with the STATGRAPHICS Plus version 4.1 software Manugestics, Inc., Rockville, Md., U.S.A.). The results were presented such as averages±SD; p<0.05 is considered as significant.

Table 3 below shows the pharmacokinetic parameters of canrenone obtained for each dose of spironolactone of 0.8 mg/kg, 2 mg/kg and 8 mg/kg. The apparent clearance for each dose (Cl_{canrenone}) was 26±8 L/kg/h⁻¹.

TABLE 3

| | Doses (mg/kg) | | |
|---|---|---|---|
| Parameters | 0.8 | 2.0 | 8.0 |
| AUC_{inf} | 426.6 ± 306.7 | 1099.0 ± 358.3 | 4794.2 ± 1393.4 |
| (μg · h · L⁻¹) | | | |
| Cmax (μg/L) | 30.9 ± 18.3 | 74.7 ± 23.9 | 261.9 ± 53.8 |
| Tmax (h) | 5.0 ± 2.17 | 5.6 ± 2.8 | 6.0 ± 1.6 |
| Cmin (μg/L) | 15.926 ± 4.290 | 16.951 ± 8.380 | 177.048 ± 42.495 |
| Clearance | 26.1 ± 8.36 | 25.7 ± 8.0 | 23.0 ± 6.2 |
| (L/kg/h) | | | |

AUC_{inff}= is the total surface area below the time-related canrenone concentration curve calculated according to the trapezoidal rule. Cmax is maximum plasmatic concentration of canrenone; Tₘₐₓ= is the time where the plasmatic concentration of canrenone is maximum.

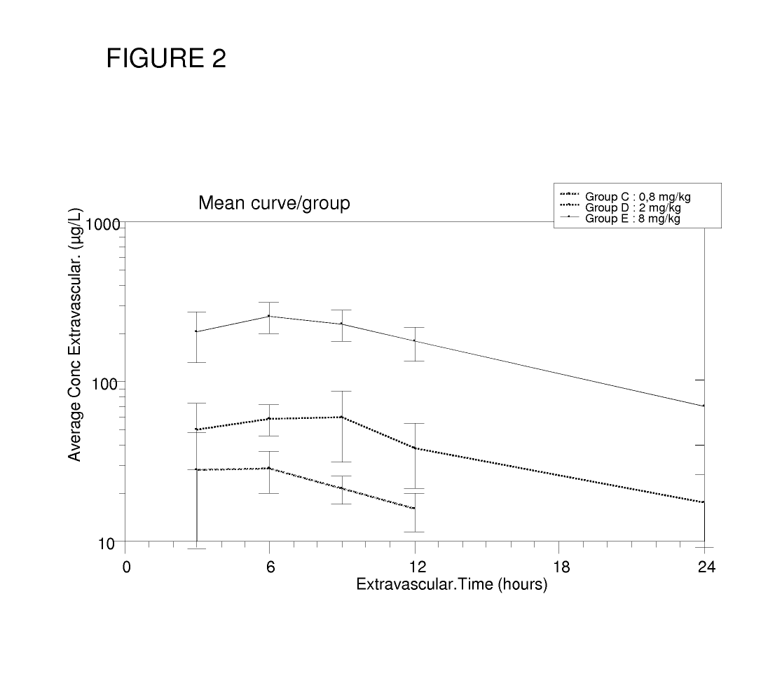

FIG. 2 represents a semi-logarithmic graph of the time-related plasmatic concentration of canrenone after oral administration of canrenone in doses of 0.8 mg/kg, 2 mg/kg and 8 mg/kg to 15 dogs. The concentration of canrenone was detected up to 5-6 hours after administration. These three curves showed parallel terminal slopes. The values of AUC_{canrenone} were 427±307, 1099±358 and 4794±1393 μg.h.L⁻¹. For the Cmax, the corresponding values were 30.9±18.3, 74.7±23.9 and 261.9±53.8 μg/L.

The doses of 0.8 mg, 2 mg/kg and 8 mg/kg of spironolactone were used in this experiment, and the dose inhibiting the effect of aldosterone on the log([Na⁺]_{urinary}×10/[K⁺]_{urinary}) was observed for the doses of 2 mg/kg and 8 mg/kg which enabled complete reversal of the effect during the first 6 hours and 12 hours after dosage. Aldosterone on its own reduced the elimination of Na by approx. 65% and the urinary levels of K were increased by 25%. Spironolactone increased the Na/K ratios after aldosterone treatment. In average, the log ([Na⁺]_{urinary}×10/[K⁺]_{urinary}) was reduced from 0.70±0.22 to 1.14±0.18 in the urine samples collected 0 to 6 hours after administration of spironolactone. The natriuretic responses were completely reversed for a dose of spironolactone of 2 mg/kg, whereas the dose of 0.8 mg/kg has no effect on certain dogs. Moreover, other increases in the elimination of Na were observed for higher doses of spironolactone (8 mg/kg).

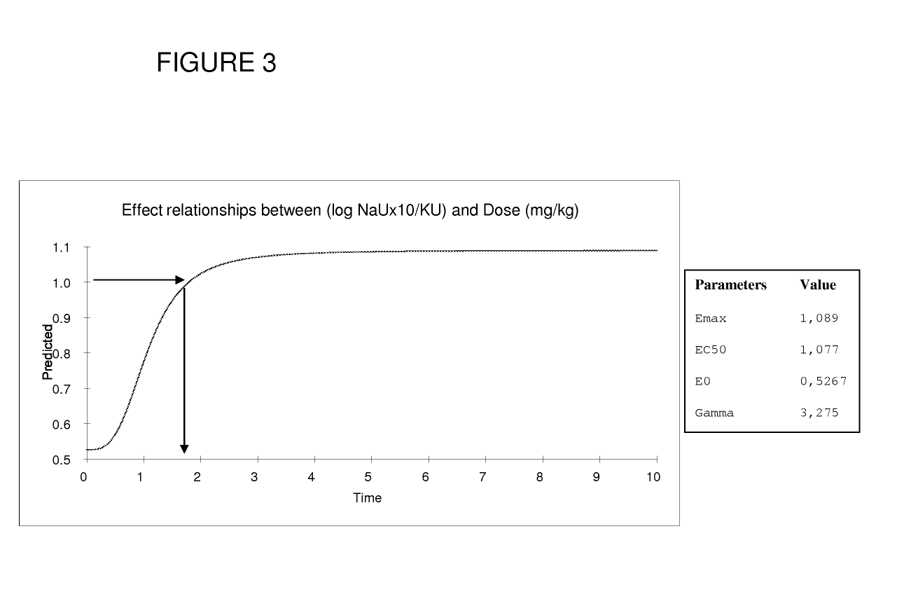

FIG. 3 represents the dose-effect relation between the doses of spironolactone and the ratio ([Na⁺]_{urinary}×10/[K⁺]_{urinary}). FIG. 3 shows the existence of a dose-effect relation by using the model (Equation 3). The value ED₅₀ after administration of spironolactone was 1.09 mg/kg. The value Eₘₐₓ (i.e. the maximum possible effect of spironolactone) was 1.089 and the value E₀ (control group was 0.527. The Eₘₐₓ−E₀ was of 0.5625 and corresponded to a 100% amplitude. Consequently, the dose of spironolactone required for restoring the Na⁺/K⁺ ratio in urines in dogs having received aldosterone et hence in a situation similar to heart failure was a dose of approx. 2 mg/kg (i.e. E_{(2mg/kg)}−E₀=0.4933) and corresponded to restoring 88% of the effect, whereas the dose of 0.8 mg/kg corresponds to 57% (i.e. E_{(0.8mmg/kg)}−E₀=0.3233). The value ED₅₀ of 1.08±0.28 mg/kg was calculated from the model of Eₘₐₓ. The efficient therapeutic dose capable of restoring and normalising the ratios was 1.80 mg/kg per day and corresponds to restoring 88% of the effect. A majority of dogs responded positively to a treatment with spironolactone administered on the basis of approx. 2 mg/kg per day.

### Example 2

Clinical studies were conducted on dogs affected by heart failure for assessing the long-term effects (14-15 months and 3 years) spironolactone-containing treatments in a dose of 2 mg/kg/day, as well as a CEI (such as for instance benazepril chlorhydrate or enalapril, etc.).

Multicentre, randomised, double-blind placebo-controlled clinical studies were conducted. An example of study concerned 221 dogs, the diagnosis of heart failure relying on persisting symptoms of cardiomegaly or cardiomyopathy after a first CEI treatment. Out of 221 dogs, 109 received orally a daily dose of 2 mg/kg/day spironolactone in the form of pellets of 10 mg, 40 mg and/or 80 mg in combination with a CEI (for instance benazepril chlorhydrate in a dose of 0.25 mg/kg/day). The 112-dog placebo group received a placebo in combination with a CEI (for instance benazepril chlorhydrate in a dose of 0.25 mg/kg/day).

For gauging the effects of the treatment, both groups were examined five times, i.e., on the first day of treatment (D1), then on the 84^{th} day (D84), 162^{nd} day (D162), 252^{nd} day (D252) and 336^{th} day (D336). This examination consisted of a clinical examination of the dogs, urine and blood analyses, and a radiograph. Moreover, an echocardiograph was taken on days D1, D168 and D336.

The efficiency and the absence of toxicity of administered doses of 2 mg/kg/day of spironolactone in combination with a standard therapy to dogs affected by heart failure were put in evidence in comparison with standard treatments on their own by the mortality rates and the mortality-morbidity rates, the latter encompassing all events such as death, euthanasia or severe deterioration in the dogs' condition. Also, the effects of the treatment were assessed on the symptoms such as cough and mobility, as well as on the prevention of symptoms such as dyspnoea, pulmonary edema, and tolerance to effort. The results obtained over 15 months were mentioned in Table 4.

TABLE 4

| | | “survival” | Survival |
|---|---|---|---|
| | | probability | probability |
| | Number of | (absence of | (absence of |
| Groups treated | dogs | morbi-mortality) | mortality) |
| Spironolactone (2 mg/kg/day) + CEI | 109 | 84% | 91% |
| (for instance 0.25 mg/kg/day benazepril | | | |
| chlorhydrate) | | | |
| Placebo + CEI (for instance 0.25 | 112 | 67% | 74% |
| mg/kg/day benazepril chlorhydrate) | | | |
| Total | 221 | | |

Other long term clinical studies have been conducted and have also shown a significant difference in the survival probabilities (absence of mortality and/or of morbidity) between both these groups of dogs treated by spironolactone+standard therapy and the standard therapy group (reference group). The results of these different studies were presented in FIGS. 2 to 6.

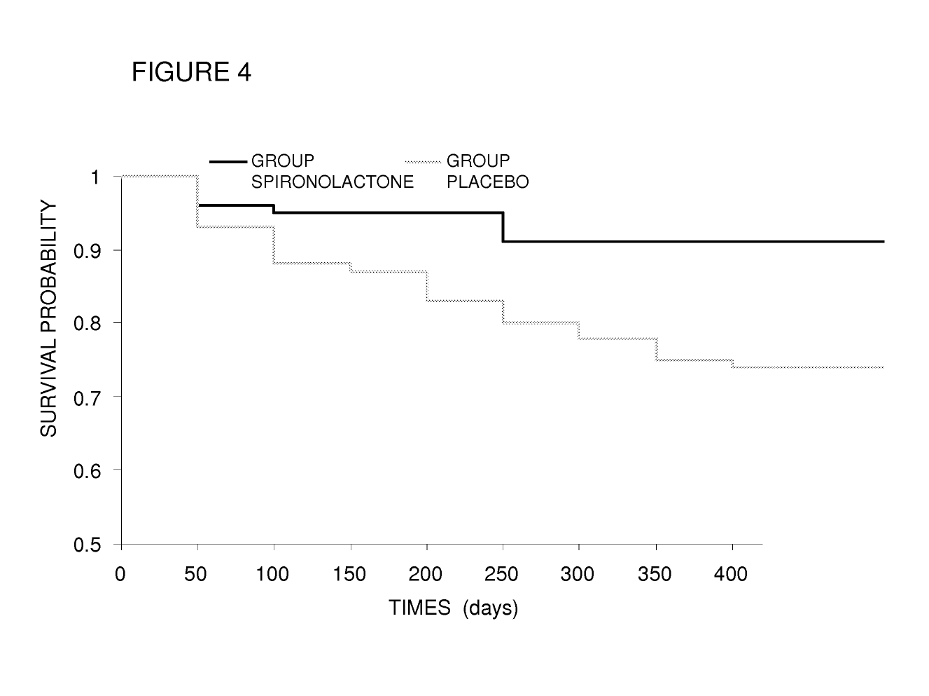

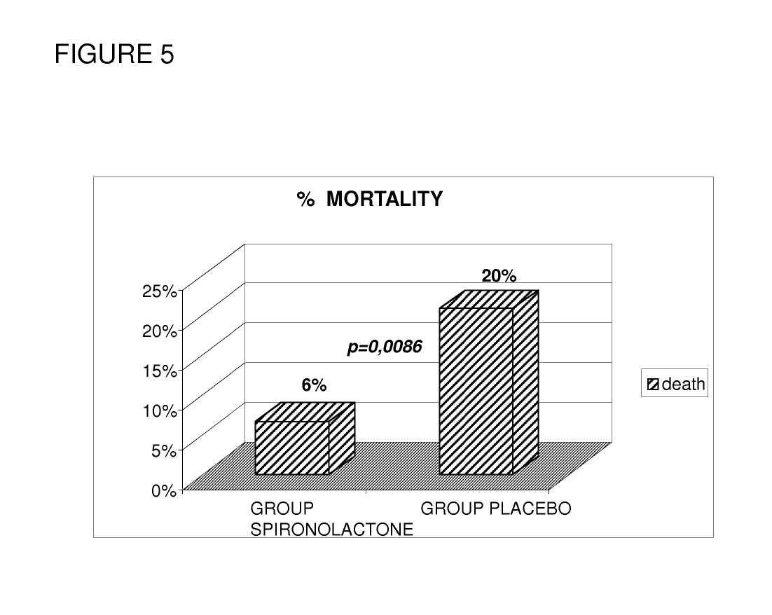

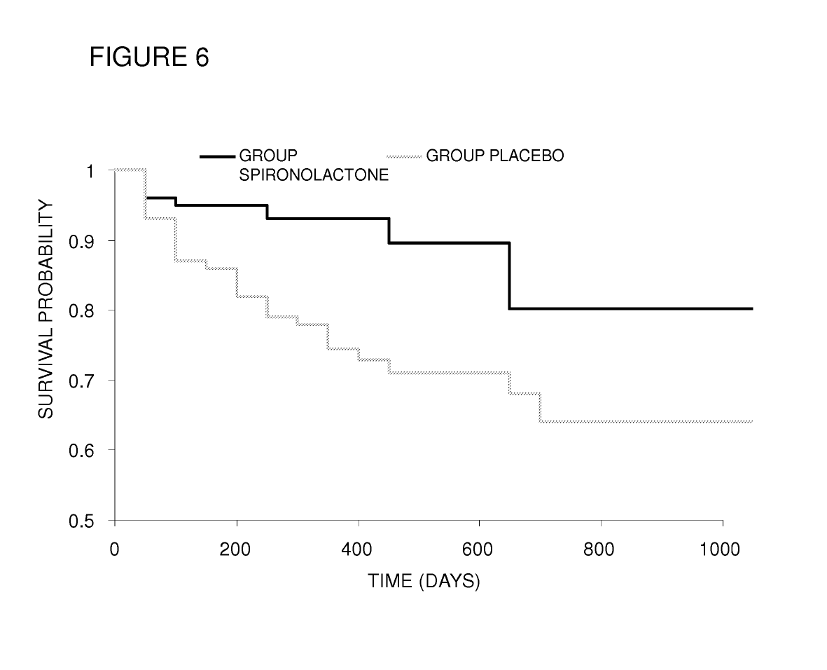

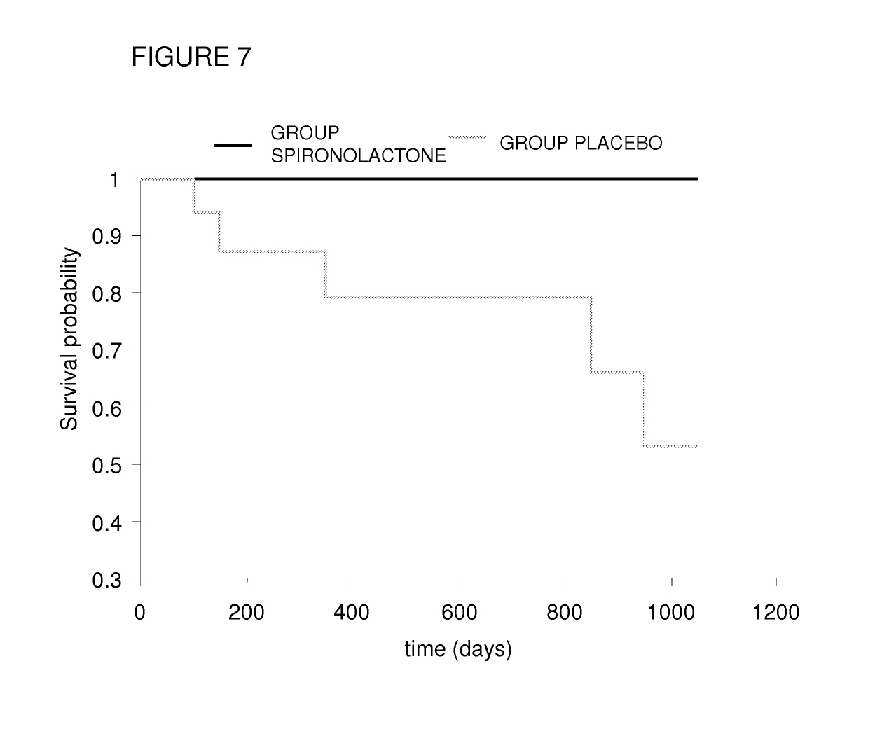

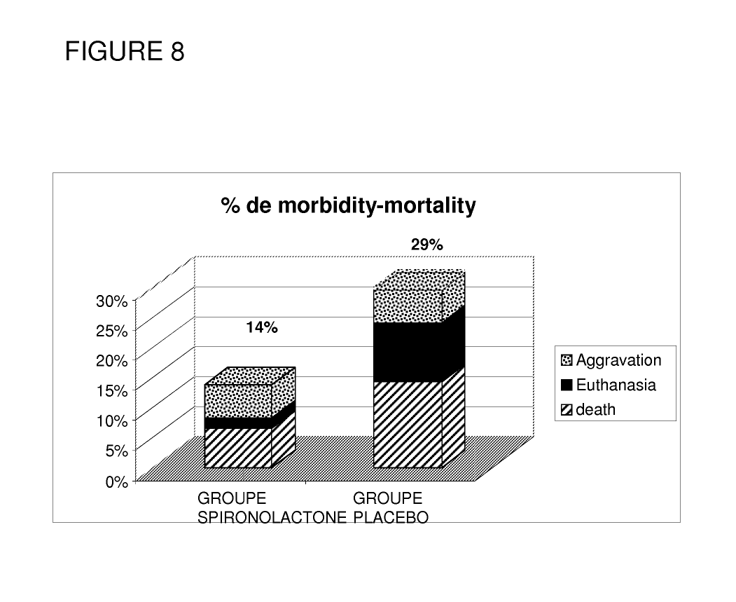

FIG. 4 illustrates the survival probabilities of dogs treated for a duration of 14 to 5 months with 91% against 74% for the reference group (p=0.11); FIG. 5 illustrates the mortality rates obtained after 14-15 months, i.e. 6% against 20% for the reference group (p=0.0029); FIG. 6 illustrates the survival probabilities of dogs treated for a duration of 3 years with 80% against 64% for the reference group (p=0.017); FIG. 7 illustrates the survival probabilities of dogs treated as of stage I of heart failure consecutively to valvular failure for a duration of approx. 3.5 years with 100% against 53%, (p=0.033); and FIG. 8 illustrates the 14-15 month morbidity-mortality rates, 11% against 25%.

Moreover, improved symptoms such as cough and mobility were observed in the group of dogs on spironolactone, as well as the prevention of symptoms of dyspnoea, pulmonary edema, tolerance to effort, and a significantly lighter deterioration in cough and syncopes. The control group showed stronger and more frequent deterioration of all these clinical signs.

### Example 3

The plasmatic potassium concentrations (mmol/L) were measured during the treatments. It has thus been demonstrated that the daily dose of spironolactone of 2 mg/kg/day which is normally a diuretic dose in men and dogs, did not cause any variation in kaliemia or only low transient variations in kaliemia in dogs. The results of the kaliemia measurements made during the clinical studies described previously have been given in Table 5 below. Only sporadic case of low or moderate hyperkaliemia could be observed and these events were transient. Indeed, these hyperkaliemia events could only be observed a couple of times during examinations, and some of them were present on day D1, before the beginning of the treatment.

TABLE 5

| | Low and transient | Moderate and |
|---|---|---|
| | hyperkaliemia | transient |
| | (5.9 to 6.4 mmol/L) | hyperkaliemia |
| | GROUP ON | (6.5 to 7.5 mmol/L) |
| | SPIRONOLACTONE | PLACEBO GROUP |
| 3-month clinical study | 5 | 210 |
| (6 analyses for the | | |
| duration of the study) | | |
| 2-month clinical study | 3 | 112 |
| (4 analyses for the | | |
| duration of the study) | | |
| 12-month clinical study | 3 | 212 |
| (5 analyses for the | | |
| duration of the study) | | |
| | 11/109 (10.1%) | 5/112 (4.5%) |
| | | 3/109 (2.8%) |
| | | 4/112 (3.6%) |

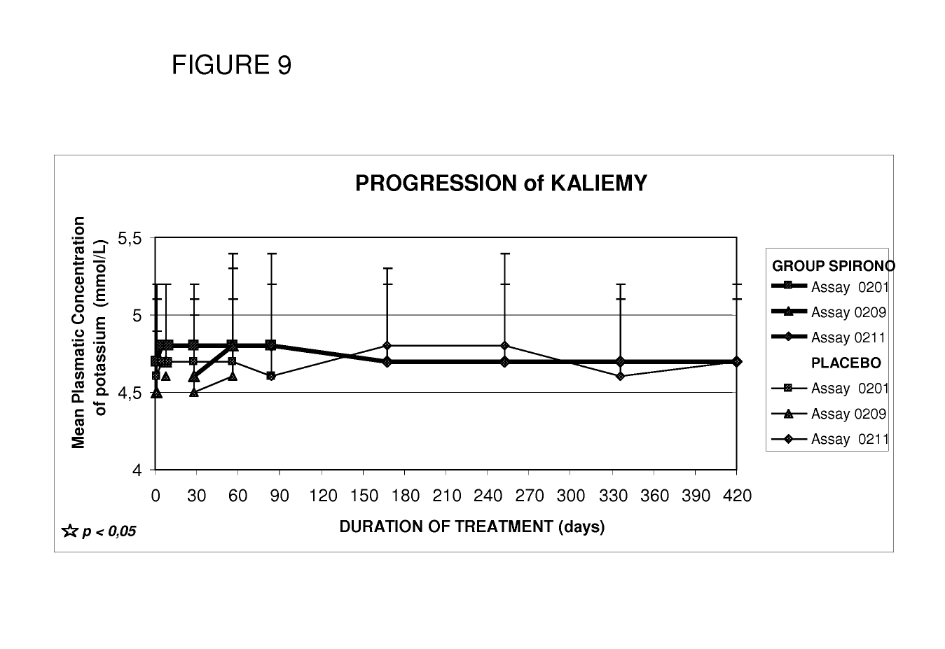

Also, FIG. 9 illustrates stable kaliemia in both groups of dogs, treated and placebo, with a few seldom cases of low or moderate hyperkaliemia and of transient nature.

1-18. (canceled)

19. A method of treating and/or preventing non-human mammal subjects affected by heart failure comprising administering an aldosterone antagonist, and a pharmaceutically acceptable vehicle to said subject, wherein said aldosterone antagonist is administered in an efficient therapeutic dose ranging between 1.5 and 5 mg/kg/day, 1.8 and 5 mg/kg/day, 1.5 and 4 mg/kg/day, or 1.5 and 3 mg/kg/day in a single take.

20. The method of claim 19, wherein said aldosterone antagonist is administered in an efficient therapeutic dose ranging between 2 and 5 mg/kg/day, in a single take.

21. The method of claim 20, wherein said aldosterone antagonist is administered in an efficient therapeutic dose of approximately 2 mg/kg/day in a single take or 4 mg/kg/day in a single take.

22. The method of claim 19, wherein said aldosterone antagonist is the spironolactone, the eplerenone, a derivative or a metabolite thereof.

23. The method of claim 19, wherein said aldosterone antagonist is administered to said subject in combination with at least one standard therapy for the treatment of heart failure.

24. The method of claim 23, wherein said standard therapy is an angiotensin converting enzyme inhibitor, an angiotensin II AT-1-receptor antagonist, an inotrope, an inodilator, a vasodilator, a diuretic, a digitalic drug, a beta blocker or a calcic antagonist.

25. The method of claim 24, wherein the angiotensin converting enzyme inhibitor is present in an efficient therapeutic dose of approximately 0.1 to 0.6 mg/kg/day, and preferably of approximately 0.25 to 0.5 mg/kg/day.

26. The method of claim 24, wherein the angiotensin converting enzyme inhibitor is alacepril, benazepril, captopril, cilazapril, delapril, enalapril, enalaprilat, fosinopril, fosinoprilat, imidapril, idrapril, lisinopril, perindopril, quinapril, ramipril, saralasin acetate, perindropilat, temocapril, trandolapril, ceranapril, moexipril, quinaprilat, spirapril, a salt or a pharmaceutically acceptable ester thereof.

27. The method of claim 26, wherein benazepril is administered to said subject in a dose of 0.25 mg/kg/day and enalapril in a dose of 0.5 mg/kg/day.

28. The method of claim 24, wherein the inotrope or inodilator is the pimobendane or the levosimendane.

29. The method of claim 28, wherein pimobendane or levosimendane is present in an efficient therapeutic dose.

30. The method of claim 29, wherein claim pimobendane or levosimendane is administered orally or by injection.

31. The method of claim 30, wherein said aldosterone antagonist is spironolactone and is administered to said subject in a dose of 2 mg/kg/day only in a single take.

32. The method of claim 31, wherein the spironolactone is administered to said subject in combination with an efficient quantity of benazepril or enalapril.

33. The method of claim 32, wherein benazepril is administered in a dose of 0.25 mg/kg/day and enalapril is administered in a dose of 0.5 mg/kg/day.

34. The method of claim 31, wherein the spironolactone is administered to said subject in combination with an efficient quantity of pimobendane or levosimendane.

36. The method of claim 19, wherein said subject affected by heart failure is a dog, a cat or a horse.

37. The method of claim 19, wherein administration is performed via oral, nasal, intradermic, cutaneous, or parenteral route.

38. The method of claim 19, wherein an aldosterone antagonist and pharmaceutically acceptable vehicle is administered is in the form of a liquid solution, suspension, solid or semi-solid, powders, pellets, capsules, granules, sugar-coated pills, gelules, sprays, cachets, pills, tablets, pastes, implants or gels.
